(19) United States
(12) Patent Application Publication
(10) Pub. No.: US 2013/0102066 A1
(43) Pub. Date: Apr. 25, 2013

(54) CHALCOGENIDE-FIBRE, INFRARED EVANESCENT WAVE SENSOR AND PROCESS FOR PRODUCING SAME

(71) Applicants: Bruno Bureau, Cesson (FR); Catherine Boussard, Betton (FR); Olivier Loreal, Bruz (FR); Patrick Houizot, Guignen (FR); Jean-Luc Adam, Rennes (FR); Jacques Lucas, Betton (FR)

(72) Inventors: Bruno Bureau, Cesson (FR); Catherine Boussard, Betton (FR); Olivier Loreal, Bruz (FR); Patrick Houizot, Guignen (FR); Jean-Luc Adam, Rennes (FR); Jacques Lucas, Betton (FR)

(73) Assignees: Centre National de la Recherche Scientifique(CNRS), Paris (FR); UNIVERSITE DE RENNES 1, Rennes (FR)

(21) Appl. No.: 13/638,929

(22) PCT Filed: Mar. 31, 2011
(86) PCT No.: PCT/EP2011/055038

§ 371 Date: Dec. 12, 2012

(30) Foreign Application Priority Data
Apr. 1, 2010 (FR) 10 52457

(51) Int. Cl. A61B 5/00; G01N 21/17

(52) U.S. Cl. CPC A61B 5/0086; G01N 21/17
USPC 435/288.7; 600/473; 156/199

(57) ABSTRACT

The invention relates to a fibre sensor that enables the propagation of infrared light at at least one wavelength of 0.8 to 25 micrometres, the fibre successively comprising along its length a first infrared waveguide section (23), a second detection section (25) intended to come into contact with an external environment in order to detect infrared signatures interfering with the propagation of the evanescent waves propagating along the fibre (2), and a third infrared waveguide section (27). The invention is characterized in that, in the second fibre section (25) that has the detection role, the fibre (2) is constituted of a curved part, the radios of curvature of which is locally less than 2.3 millimetres.

The invention concerns a chalcogenide fiber sensor using evanescent infrared waves.

Chalcogenide glass is glass with a low melt temperature and low glass transition temperature and such glasses are highly original materials, difficult to synthesize and control. Their performance greatly extends the sensitivity of Fiber Evanescent Wave Spectroscopy sensors (FEWS). More particularly, these glasses containing sulfur, selenium and/or tellurium allow light to pass over a broad wavelength range in the infrared, which is not the case for conventional oxide glasses. In addition, the glassy nature of the material allows the forming thereof to fabricate optical fibers. Finally, they are glasses which entail chemical elements that are close to one another in the periodic table which therefore maintain highly covalent bonds between each other. This nature of the chemical bonds makes the material hydrophobic which is most positive when they are used as sensor in media with high water content such as biological samples.

One of the fields of application of this sensor is effectively the biomedical field.

The document “IR optical fiber sensor for biomedical applications” by J. Keirsse, C. Boussard-Plédel, O. Loréal, 0. Sire, B. Bureau, P. Leroyer, B. Turlin, J. Lucas, Vibrational Spectroscopy 32 (2003), pages 23 to 32, describes the principle of said sensor in the mid-infrared range. In this device, a single Te₂As₃Se₅ fiber is used as waveguide and as detection element. This fiber has a spectral window ranging from 800 to 4,000 cm⁻¹. To improve the sensitivity of this sensor, its diameter is locally reduced to create a tapered U-shaped detection zone which is brought into contact with a sample to be analyzed. The diameter of this fiber of 400 micrometers is reduced to 100 micrometers in this detection zone. The fiber is coupled to a FTIR spectrometer. The principle of measurement is to send an infrared wave into the fiber which propagates along the fiber via successive reflections on its outer surface. In the detection zone in contact with the sample, the evanescent wave propagating along the outer wall of the fiber is partly absorbed by the target substance. The spectrometer analyzes the wave received at the other end of the fiber to deduce information on the sample therefrom.

The document “Advances in chalcogenide, fiber evanescent wave biochemical sensing” by Pierre Lucas, Mark R. Riley, Catherine Boussard-Plédel, Bruno Bureau, Analytical Biochemistry (2006), pages 1 to 10, describes a chalcogenide fiber sensor specifying that the radius of curvature of the fiber depends on its diameter and may reach 2 centimeters for a 100 micrometer fiber.

The problems raised by the sensor are its lack of compactness, fragility, low sensitivity in the detection zone.

The invention sets out to obtain a sensor using at least one fiber in chalcogenide glass operating on the principle of absorption via evanescent waves that is compact, robust and sensitive to signatures in the mid-infrared of the target molecules.

The first subject of the invention is a sensor meeting these criteria, comprising at least one fiber allowing the propagation of infrared light at least at one infrared wavelength of between 0.8 and 25 micrometers, and outwardly generating evanescent waves to detect infrared signatures of an outside medium, said at least one fiber being of composition of XY type where X is chosen from among Ge, As or Sb or a mixture of two or more than two of these components, and where Y is chosen from among S, Se, Te or a mixture of two or more than two of these components,

the fiber successively comprising over its length a first fiber section for guiding the infrared wave, at least one second fiber section having a detection function and intended to come in contact with the outside medium to detect infrared signatures perturbing the propagation of evanescent waves propagating along the fiber, and a third fiber section for guiding the infrared wave,

characterized in that

in the second fiber section having the detection function, the fiber is formed of at least one bent part whose radius of curvature is locally smaller than 2.3 millimeters.

Surprisingly, the sensitivity of the sensor is increased at the contact zone by means of this small radius of curvature.

According to one embodiment of the invention the mechanical compressive strength of the bent part of the second fiber section (25), in the direction in which it is sought to draw together two separate points belonging to the bent part, that is equal to or higher than 1 N.

According to one embodiment of the invention, said at least one bent part has a fiber radius of curvature equal to or less than 1 millimeter.

According to one embodiment of the invention, the first fiber section (23) and the third fiber section are distant from one another by a width of less than 2.8 mm transverse to the length of the fiber and occupy a space of width less than 2.8 mm transverse to the length of the fiber.

According to one embodiment of the invention, the bent part (28) comprises a winding.

According to one embodiment of the invention, the bent part (28) comprises a winding comprising at least one turn,

the bent part of the second fiber section (25) having a mechanical compressive strength, in the direction in which it is sought to draw together two separate points belonging to the bent part, equal to or higher than 1 N per turn.

According to one embodiment of the invention, at least one transverse dimension (D1) of the fiber in the winding is smaller than at least one transverse dimension (D2) of the fiber in the first and third sections.

According to one embodiment of the invention, the second fiber section has a fiber diameter (or thickness D2) of between 50 and 450 micrometers.

According to one embodiment of the invention, on the first, second and third sections (23, 25, 27), the fiber has a thickness of between 50 and 450 micrometers that is constant over these first, second and third sections (23, 25, 27).

According to one embodiment of the invention, the first and third fiber sections are inserted in a protective sheath, the second section projecting at least partly from one end of the sheath and being intended to come into contact with the medium being examined, whether this is solid and/or liquid and/or gaseous.

According to one embodiment of the invention, the first and third fiber sections are inserted in an operative channel of a medical diagnostic device, the second section projecting at least partly from one end of the channel and being intended to come into contact with a tissue or a biological fluid in vivo and/or ex vivo.

According to one embodiment of the invention, the proportion of X by weight is equal to or higher than 10% and equal to or lower than 70%, whilst the proportion of Y by weight is equal to or higher 30% and equal to or lower than 90%.

A second subject of the invention is a method for fabricating a said sensor such as described above, characterized in that

the chalcogenide fiber has a composition of type XY where X is chosen from among Ge, As or Sb or a mixture of two or more than two of these components, and where Y is chosen from among S, Se, Te or a mixture of two or more than two of these components, the chalcogenide fiber having a glass transition temperature T_{g}, the fiber successively comprising over its length—between two first and second ends of the fiber—the first fiber section, an intermediate part which is to form the second fiber section and the third fiber section,

a core is heated having a contact zone of transverse dimensions smaller than 4.6 millimeters, to a certain temperature, and the intermediate part of the fiber is applied against said contact zone of the core so that the intermediate part in contact with the core zone has a temperature T₂ with:

1.05·T_{g}≦T₂≦1.5·T_{g},

the intermediate part of the fiber is wound around the contact zone of the core at a winding angle of at least 180° so as to form said bent part in said intermediate part of the fiber thereby forming said second fiber section with a radius of curvature locally smaller than 2.3 millimeters.

According to one embodiment of the invention, the core comprises first and second portions between which said contact zone is located, the core being heated by at least one of the first and second portions.

According to one embodiment of the invention, the core is cylindrical having any cross-section.

According to one embodiment of the invention, the temperature T₂ exceeds the glass transition temperature T_{g} by 10% to 20%.

According to one embodiment of the invention, the intermediate part has a fiber diameter (D2) of between 50 and 450 micrometers.

The invention will be better understood on reading the following description given solely as a non-limiting example with reference to the appended drawings in which:

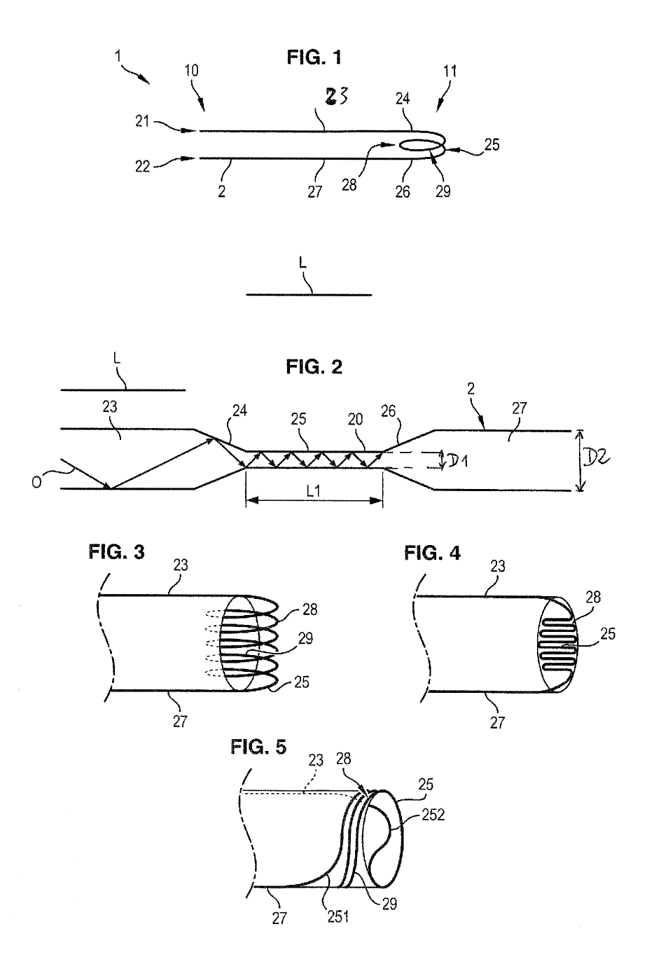

FIG. 1 schematically illustrates a magnified perspective view of a first embodiment of a sensor according to the invention,

FIG. 2 is a schematic, longitudinal section view of the detection zone of the sensor of the invention before bending, the intermediate part having a diameter which may be equal to the diameter of parts 1 and 3,

FIGS. 3, 4 and 5 are magnified, schematic perspective views of second, third and fourth embodiments of the sensor according to the invention,

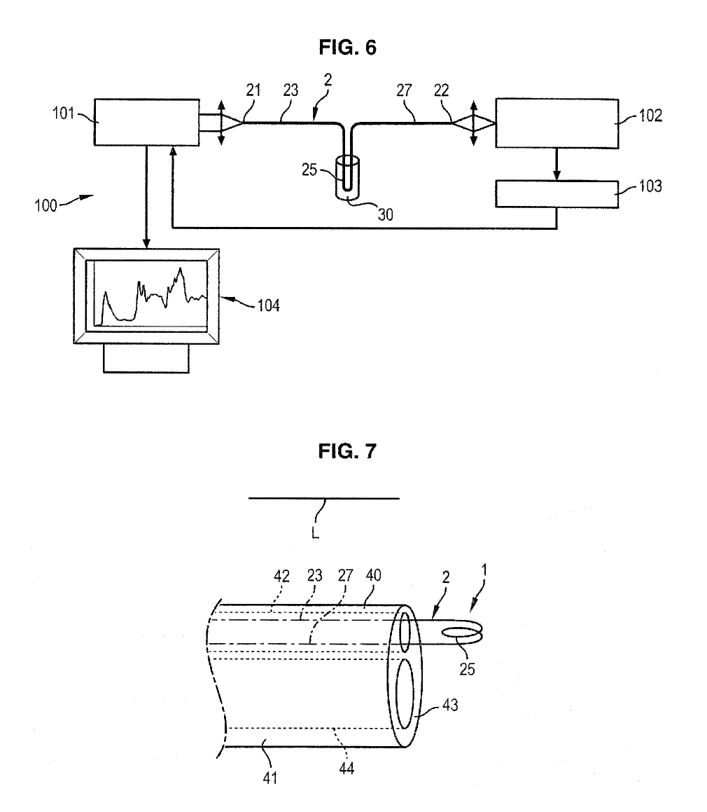

FIG. 6 schematically illustrates a device using the sensor of the invention,

FIG. 7 is a magnified, perspective schematic view of an endoscopic tube using the sensor of the invention,

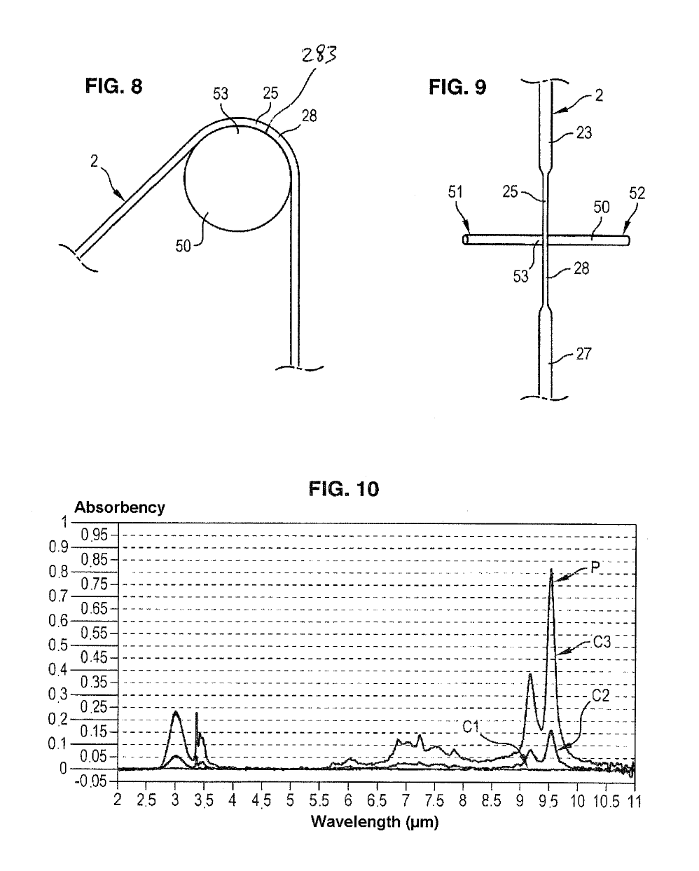

FIG. 8 is a magnified schematic side view of a fiber of the sensor according to the invention at one step of its method of fabrication,

FIG. 9 is a magnified, schematic overhead view of a fiber of the sensor according to the invention at the step of its method of fabrication in FIG. 8,

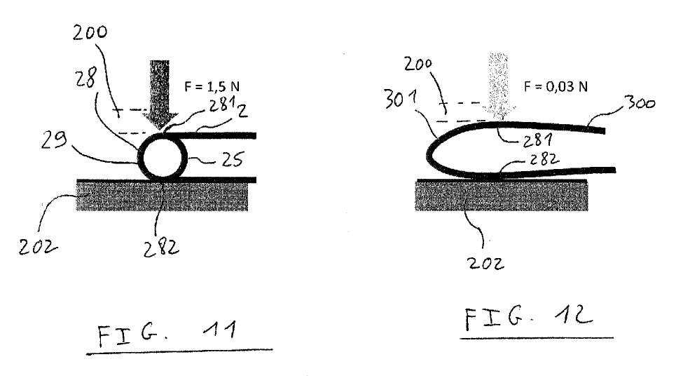

FIG. 10 is a diagram of the absorbency of one example of a fiber according to the invention as a function of wavelength.

In the Figures, the sensor 1 comprises a fiber 2 in chalcogenide glass. The fiber has a composition of XY type where X is Ge, As or Sb or a mixture of two or more than two of these components, and where Y is S, Se, Te or a mixture of two or more than two of these components. For example, the proportion of X by weight is equal to or higher than 10% and equal to or lower than 70%, whilst the proportion of Y by weight is equal to or higher than 30% and equal to or lower than 90%. The fiber is used for guiding infrared waves.

In one example of embodiment, the fiber is in As₂Se₃.

In another example of embodiment, the fiber is in Te₂As₃Se₅.

The fiber 2 of the sensor 1 extends in a general longitudinal direction L between a first end 21 and a second end 22. The fiber 2, from the first end 21 to the second end 22, comprises a first fiber section 23 for guiding infrared waves, this first section 23 being connected on its side distant from the first end 21 to a first point 24 of a second detection section 25, said second section 25 is connected via a second point 26 distant from the first point 24 to a third fiber section 27 for guiding infrared waves.

In one embodiment, the second detection section 25 of the fiber 2 has a transverse width D1 in at least one dimension, a diameter D1 or cross-section D1 which are smaller than the transverse width D2 or diameter D2 or cross-section D2 of the first section 23, and which are smaller than the transverse width D2, the diameter D2 or cross-section D2 of the third section 27. The second detection section 25 of the fiber 2 extends over a nonzero length L1 of the fiber between the first and second connecting points 24, 26 and between the first section 23 and the third section 27.

For example, the fiber 2 has a mean diameter D2 of about 400 micrometers in the first section 23 and in the third section 27, whilst the fiber 2 in the second detection section 25 has a mean diameter D1 of about 100 micrometers for a length L1 of the second detection section of about 10 centimeters. The length of the first section 23 is longer than the length of the second detection section 25. The length of the third section 27 is longer than the length L1 of the second detection section 25.

The first connecting point 24 is formed for example by a zone with cross-section tapering from the first section 23 to the second detection section 25, being of truncated cone shape for example as illustrated in FIG. 2. The second connecting point 26 has a cross-section which for example widens from the second detection section 25 to the third section 27, being of truncated cone shape for example as illustrated in FIG. 2. Between the first and second connecting points 24, 26 the second detection section 25 for example has a constant transverse width D1 and/or a constant transverse diameter D1 and/or a constant cross-section D1.

In another embodiment, the second detection section 25 of the fiber 2 has a transverse width (or thickness) in at least one dimension, a diameter or a cross-section which are equal to the transverse width or the diameter or the cross-section of the first section and/or which are equal to the transverse width (or thickness), the diameter or the cross-section of the third section 27. For example, the fiber 2 has a cross-section and/or diameter that are constant in the first, second and third sections 23, 25 and 27.

In one embodiment the fiber—on the first, second and third sections 23, 25 and 27—has a fiber diameter (or thickness) for example of between 50 and 450 micrometers.

For example in one embodiment—on the first, second and third sections 23, 25 and 27—the fiber has a diameter (or thickness) of between 50 and 450 micrometers that is constant on these first, second and third sections 23, 25 and 27.

The second detection section 25 is intended to come into contact with an outside medium to detect the perturbations caused by this outside medium to the propagation of infrared waves in the fiber 2, and forms a detection head of the sensor. The first and second sections 23, 27 are sections conveying infrared waves.

FIG. 2, via thick arrows inside the fiber 2, symbolically shows the pathway of an infrared wave O sent by the first end 21 into the section 23. On account of the narrowing of the cross-section of the fiber 2 in the second detection section 25, there are more reflections of the wave in this second section 25 against the outer surface 20 of the fiber 2.

When an outside medium is contacted with the second detection section 25, the propagation of the wave O in this second section 25 is perturbed since part of the wave O passes from the second section 25 towards the outside medium.

As a result, the contacting of the second detection section 25 with an outside medium will have an influence on the wave O which is transmitted from the first section 23 to the third section 27.

According to the invention, the second detection section 25 comprises at least one bent fiber part 28 having a fiber radius of curvature smaller than 2.3 millimeters.

The bent part 28 is in the form of a winding for example having one or more turns, or may be of solenoid shape for example. In another example the bent part 28 may be U-shaped. The second section is in the form of an open loop for example. Evidently it is possible to have any number of bent parts 28 e.g. n open loops of any geometry, n≧1.

In FIG. 2, the second detection section 25 is illustrated rectilinear fashion before being bent to form the bent part 28.

For example, after bending, the sections 23 and 27 face one another. For example, the sections 23 and 27 are parallel to each other.

For example, the radius of curvature of the bent part 28 of the second section 25 is equal to or smaller than 1.4 mm, and in particular equal to or smaller than 1 mm.

For example, in the embodiments shown in the Figures, the first fiber section 23 and the third fiber section 25 are spaced apart by a width of less than 2.8 mm transverse to the length of the fiber and occupy a space of width smaller than 2.8 mm transverse to the length of the fiber.

Some embodiments of the second section 25 are illustrated in FIGS. 1, 3, 4 and 5.

In the embodiments shown FIGS. 1, 3 and 5, the bent part 28 is shaped in winding having one or more turns 29 circular for example.

In the embodiment shown FIG. 1, the bent part 28 is formed of one and a half turns 29 and lies more or less in the general plane of the sections 23 and 27, the winding 28 being illustrated in expanded form at the top of FIG. 1 to show the different parts of the winding 28. In general, the turn(s) 29 of the winding may each lie in a plane.

In the embodiment illustrated FIG. 3, the turns 29 of the winding of part 28 are of general helical shape connected to sections 23 and 27 and face each other, and may for example lie parallel to one another.

Provision could also be made for single helical turn, or one and a half helical turns 29.

In the embodiment illustrated FIG. 4, the bent part 28 forms meanders changing convexity at least once e.g. in one plane.

In the embodiment illustrated FIG. 5, the bent part 28 comprises a winding having one or more turns 29 turning around a geometric cylinder along which the sections 23 and 27 are positioned, the geometric cylinder on which the winding is wound being circular for example. The connecting parts 251, 252 of the turns 29 to the sections 23 and 27 also have a radius of curvature smaller than 2.3 millimeters which is of 0.8 mm for example for a radius of curvature of about 1 mm of the turns 29 around the geometric cylinder. The diameter of the geometric cylinder around which the fiber 2 is wound is between 1.5 and 4 mm.

A method for obtaining the bent part 28 in the second detection section 25 shown FIG. 2 is described below with reference to FIGS. 8 and 9.

The tapering 24, 26 is optional when forming the head 28. To fabricate the sensor 1 of the invention, a chalcogenide glass fiber is drawn to a determined mean diameter e.g. 400 micrometers to form the first and third sections 23, 27. The second section 25 having a smaller transverse width than the sections 23, 27 in at least one dimension is formed for example by accelerating the rate of drawing, by chemical attack with a sulfuric acid solution and hydrogen peroxide or by hot crushing at a temperature of T_{g}+15%, T_{g} being the glass transition temperature of the material of the fiber 2. For example a section 25 is obtained able to be reduced to less than 100 micrometers in at least one transverse dimension.

The glass transition temperature T_{g} is measured by differential calorimetric analysis with a temperature rise of 10° C. per minute.

The fiber 2 then extends in the longitudinal direction L as shown FIG. 2.

The section 25 is then bent by hot forming. To do so, the section 25 of fiber 2 is applied against a heated core 50 so that the intermediate part 25 which is placed in contact with the core 50 is at a temperature T₂ such that:

1.05·T_{g}≦T₂≦1.5·T_{g},

and in particular

1.1·T_{g}≦T₂≦1.219 T_{g}.

In one example of embodiment, the glass transition temperature T_{g} is exceeded by 10% to 15% to heat the section 25 which is to be bent.

The contact zone 53 of the core 50 against which the section 25 is applied has transverse dimensions (e.g. diameter) of less than 4.6 mm for the winding of the section 25 in accordance with the radius of curvature imposed by this contact zone 53. For example, the contact zone 53 of the core 50 has transverse dimensions (e.g. diameter) equal to or less than 2.4 mm, and for example equal to or less than 2 mm. The transverse dimensions or diameter of the zone 53 around which the section 25 of the fiber 2 is wound are between 1.5 and 4 mm for example.

In general for the chalcogenide glass used, the glass transition temperature T_{g} is equal to or higher than 90° C., and equal to or lower than 400° C.

The heating of the core 50 takes place for example in two first and second portions 51, 52 thereof, between which there is a third contact zone 53 against which the intermediate section 25 of the fiber 2 is applied. The heating of this third zone 53 takes place by thermal conduction for example from the portions 51, 52, the core being metallic for example, means for heating the core 50 being provided. The core 50 is of oblong shape for example between the portions 51, 52. At least in its contact zone 53, or entirely, the core 50 is of cylindrical shape for example, in particular circular cylindrical. Evidently, the core 50 could solely be heated from only one of the two portions 51, 52.

To achieve bending, the intermediate section 25 is applied against the core 50 thus heated and having the desired radius of curvature. This core is in stainless steel for example and is of circular cross-section for example to form a circular winding in the section 25. In this manner, the section 25 is wound in controlled manner around the heated core 50 to form the bent part, for example by one or more turns of the winding 28 i.e. turning by at least 360° around the core 50, the bent part 28 therefore being bent with the radius of curvature of the core 50.

The section 25 is then cooled in controlled manner, for example at 2° C. per minute and the core 50 is then removed.

In this manner, several bent parts 28 were fabricated formed of a winding having between one and five turns plus one half turn according to FIG. 1, having a radius of curvature of 1 mm in the section 25 with a fiber diameter D1 of 100 micrometers, the sections 23 and 27 having a fiber diameter D2 of 400 micrometers, the fiber having a circular cross-section. The preferred model is a model with three turns, beyond this amount the length of the solenoid (dimension parallel to the axis of symmetry) becomes greater than the diameter of the turns and affects the compactness of the assembly.

Therefore it is the core 50—around which the fiber is wound—that is heated to transmit its heat to the fiber. This provides a chalcogenide fiber sensor having better mechanical strength in the bent part 28 since the inner surface 283 of the bent part 28 which has been heated undergoes annealing via this localized heating.

The hot forming obtained by this localized heating via the core 50 reinforces the mechanical strength of the chalcogenide fiber sensor significantly by removing the residual stresses generated during the bending of the fiber. For example, the inventors have determined a strength value of 4 Newtons for a bent part 28 with three turns having a fiber diameter of 200 μm and a radius of curvature of 1 mm as in FIG. 1 (bent part 28 formed by a winding of three and half turns).

In one embodiment, the heated core 50 around which the fiber is wound comprises a core in copper or stainless steel coated with a layer of Teflon to prevent the fiber from adhering to the core 50. In general, the core 50 in a first metal material is coated with an outer layer in a different second material which is anti-adhesive for the fiber e.g. Teflon.

The manner used to measure the mechanical strength of the bent part 28 of the chalogenide fiber according to the invention is the same as illustrated in FIG. 11 and is described below.

In FIG. 11 the measurement of mechanical strength is carried out on a fiber 2 of the invention whose bent part 28 is formed of one and a half turns (called the turn 29 in the remainder hereof) therefore being wound one and half times so that the sections 23 and 27 are parallel and face one another. To carry out this measurement, the bent part 28 is immobilized in the plane of FIG. 11 between a second jaw 202 and a first jaw 200 which applies a force F onto the bent part 28 tending to cause the jaws 200 and 202 to draw together. Therefore the turn 29 of the bent part 28 comprises a third contact point 281 with the first jaw 200 and a fourth contact point 282 with the second jaw 202, this fourth point 282 being separate from the third point 281 and for example being diametrically opposite the third point 281 in the plane of the turn. A determined compressive force F is applied to the third point 281 of the bent part 28 relative to the fourth point 282. The force F applied to the bent part 28 therefore tends to cause the two separate points 281 and 282 to draw near to each other. The mechanical compressive strength of the bent part 28 is the value of the force F beyond which the bent part 28 ruptures. Therefore, for the indicated value F of mechanical strength, the bent part 28 of the fiber does not break. The measurement of the force F is provided for example by a force sensor arranged on an actuator drawing together the jaws 200 and 202. For this measurement of mechanical strength, a compression machine was used namely a Lloyd LR50K instrument in which the travel rate of the jaw 200 towards the jaw 202 was 10 mm/minute.

For example in FIG. 11, for a fiber 2 whose bent part 28 has one and half turns 29 formed according to the fabrication method of the invention, a mechanical compressive strength was measured of F=1.5 N, this fiber 2 of the invention having the following parameters:
radius of curvature of the one and half turns 29 of the bent part 28: 1 mm;
constant thickness (fiber diameter) of the fiber over its entire length (sections 23, 25 and 27): 240 μm;
composition of the fiber 2: Te₂₀As₃₀Se₅₀;
outer dimensions (height) between points 281 and 282: 2 mm.

In the comparative example in FIG. 12, the same measurement method was applied as in FIG. 11, but on a fiber 300 representing the prior art and not formed using the method of the invention, bent to a U-shape and having the following parameters:
radius of curvature of the U-shaped bent part 301: 10 mm;
constant thickness (fiber diameter) of the fiber over its entire length (in the bent part 301 and in the fiber 300): 240 μm;
composition of the fiber 300: Te₂₀As₃₀Se₅₀;
outer dimensions (height) between points 281 and 282: 20 mm;
method for bending the fiber 300 of the prior art: the fiber 30 is bent at ambient temperature (about 20° C.) without heating and without winding.

A mechanical compressive strength was measured of F=0.03 N as shown in FIG. 12.

The sensor of the invention therefore intrinsically has a greater mechanical strength than the bent fibers of the prior art in chalcogenide.

Chalcogenide fibers of the prior art are effectively known to break easily on account of their composition.

The detection head formed by the bent part 28 of the invention is more resistant and more rigid, which allows better contact with the substrate being examined. For example, the good mechanical strength of the bent part 28 allows easy handling of the sensor, in particular for its insertion into any suitable sheath or tube or more generally in any suitable box or casing for use thereof, and also allows the bent part 28 forming the detection head to be applied and abutted with some force against a substrate of solid consistency that is to be examined to ensure that the detection part 25 is in contact with this substrate, such as a body organ or part of a living human or living animal body.

In general, the bent part 28 may have any number of n+½ turns 29 where n is a natural integer equal to or more than zero, to cause the first and third sections 23 and 27 to face one another being substantially parallel for example.

In FIG. 6 an example of a device is illustrated for the functioning of the sensor of the invention. This device comprises an infrared spectrometer 101 connected to the first end 21 of the fiber to which it sends an infrared signal. The second detection section 25 of the fiber 2 is shown in contact with a laboratory sample 30 in a test tube or other i.e. outside a human or animal body. The second end 22 of the fiber 2 is connected to an infrared detector 102 to receive the infrared signal transmitted from the first end 21 to the second end 22 via the sections 23, 25, 27 of the fiber 2. The detector 102 is connected to an amplifier 103 of the signal received by the detector 102. The signal once amplified by the amplifier 103 is sent to the spectrometer 101 comprising a signal processing unit 104 allowing the infrared signal received at the end 22 to be compared with the infrared signal sent to the end 21. This comparison allows an evaluation of the perturbation caused by the sample 30 or more generally by the outside medium on the second intermediate detection section 25.

With this detection method it is possible for example on a sample 30 to evidence metabolic anomalies which reflect a pathological condition in a serum or in hepatic biopsies, or a bacterial contamination of an organic medium.

The spectrometer 101 emits in the mid-infrared. The spectrometer comprises a spectra analysis algorithm of the sent signal and of the received signal. The sent signal may also be in the far infrared. The outside medium with which the intermediate detection section 25 is placed in contact may be solid, liquid or gaseous.

For example, the first fiber section 23 and the third fiber section 27 are spaced apart by a width of less than twice the maximum radius of curvature of the winding 28 transverse to the length of the fiber and occupy a space having a width of less than twice the maximum radius of curvature of the winding 28 transverse to the length of the fiber.

In the embodiments shown FIGS. 1, 3, 4, 5 and 7 the first and third sections 23, 37 face one another. In the embodiments shown FIGS. 1 and 3 the first and third sections 23, 37 are spaced apart by a distance equal to or less than twice the maximum radius of curvature of the second section 25 in FIGS. 1 and 3.

The sections 23 and 27 are parallel for example. The sensor 1 extends globally between a first side 10 where the first and second ends 21, 22 are positioned, and a second side 11 where the second detection section 25 is positioned, the fiber following an outward pathway from the first side 10 to the second side 11 via the first section 23, then a return pathway from the second side 11 to the first side 10 via the third section 27. Evidently, the first or second end 21, 22 may project beyond the first side 10.

In the embodiment illustrated FIG. 7, the sensor 1 such as described in FIG. 1 is inserted in a support 40. This support 40 is of elongate shape for example in the longitudinal direction L, being in the form of a sheath for example. The support 40 comprises a body 21 inside which there is a longitudinal conduit 42 in which the first and second sections 23, 27 are inserted which face each other. For example, the conduit 42 has a transverse width equal to or more than twice the radius of curvature of the second section 25 in FIG. 1 or 3, or equal to or more than twice this radius of curvature to which is added the transverse thickness of the two sections 23, 27.

The body 41 and the conduit 42 extend as far as a detection end 43. The second detection section 25 projects at least partly, for example fully as shown in FIG. 7, outside the detection end 43 outside the conduit 42. Part of the first and second sections 23, 27 may also project beyond the detection end 43. The support 40 is tube-shaped for example. The support 40 is an endoscopic tube for example to investigate inside the body of a living being. For example the endoscopic support 40 is used to bring the detection section 25 of the sensor 1 against a living tissue inside a human or animal body for investigation thereof. By pressing the detection end 43 of the tube against the living tissue inside the body of the living being, the detection part 25 of the sensor 1 is applied against this living tissue to detect any perturbations it causes to the propagation of the infrared signal in the fiber 2. Evidently, the support 40 may comprise one or more other longitudinal conduits 44. The sensor 1 in FIG. 7 could evidently be one of the other embodiments described above.

The sensor 1 described above can be inserted in the operating channel of a medical device so that the detection head 28 is in contact with a medium or biological tissue which may be solid or liquid for example, in vivo or in vitro. For example it may be inserted in a catheter, in a medical diagnosis device, inside a living being or against a living being (e.g. the skin), in a medical analysis device (e.g. in vitro blood analysis).

The sensor 1 may also be used to detect the presence in the outside medium of one or more chemical substances having an infrared signature in the spectrum of the fiber ranging from 0.8 to 25 micrometers, or to measure the quantity of such substances in the outside medium.

A test was performed on the sensor 1 in FIG. 1 having a winding 28 with a radius of curvature of 1 millimeter (diameter of 2 millimeters) forming one and a half turns i.e. a turn of 360° and a half-turn representing an equivalent length L1 of about 5 millimeters. With this solenoid sensor, the spectrum of ethanol was measured. FIG. 10 shows the absorbency along the Y-axis as a function of wavelength shown along the X-axis, for:
the fiber 2 of the sensor 1 without ethanol i.e. in air, as per the first curve C1,
the fiber 2 of the sensor 1 having its third section 27 in contact with ethanol and its section 25 in air, as per the second curve C2,
the fiber 2 of the sensor 1 having the second section 25 in contact with ethanol on its end located the closest to the side 11 i.e. on the part of the winding 25 the furthest to the right in FIG. 1.

It can be seen that the curve C3 has very high absorbency compared with the curves C1 and C2. The peak P at about 9.5 micrometers corresponds to a spectral ray of the ethanol which is therefore better detected by the sensor of the invention having the detection section 25.

By means of the high sensitivity of the detection head 28 of the invention, it is possible to detect molecular signatures in the infrared in the outside medium with which this head 28 is placed in contact, and in particular to detect the presence of molecules in smaller quantities for one same molecular formula but also a higher number of molecules having different molecular formulas.

1. A sensor having at least one fiber allowing the propagation of infrared light at least at one infrared wavelength between 0.8 and 25 micrometers and outwardly generating evanescent waves to detect the infrared signatures of an outside medium, said at least one fiber (2) having a composition of XY type where X is chosen from among Ge, As or Sb or a mixture of two or more than two of these components, and where Y is chosen from among S, Se, Te or a mixture of two or more than two of these components,
the fiber successively comprising over its length a first fiber section (23) for guiding the infrared wave, at least one second fiber section (25) having a detection function and intended to come into contact with the outside medium to detect infrared signatures perturbing the propagation of the evanescent waves propagating along the fiber (2), and a third fiber section (27) for guiding the infrared wave,
characterized in that
in the second fiber section (25) having the detection function, the fiber (2) is formed of at least one bent part whose radius of curvature is locally smaller than 2.3 millimeters.

2. The sensor according to claim 1, characterized in that the bent part of the second fiber section (25) has a mechanical compressive strength, in the direction in which it is sought to draw together two separate points belonging to the bent part, which is equal to or higher than 1 N.

3. The sensor according to claim 1, characterized in that said at least one bent part has a fiber radius of curvature equal to or smaller than 1 millimeter.

4. The sensor according to any of the preceding claims, characterized in that the first fiber section (23) and the third fiber section (27) are spaced apart by a width of less than 2.8 mm transverse to the length of the fiber and occupy a space of a width smaller than 2.8 mm transverse to the length of the fiber.

5. The sensor according to any of the preceding claims, characterized in that the bent part (28) comprises a winding.

6. The sensor according to any of the preceding claims, characterized in that the bent part (28) comprises a winding comprising at least one turn,
the bent part of the second fiber section (25) having a mechanical compressive strength, in the direction in which it is sought to draw together two separate points belonging to the bent part, which is equal to or higher than 1 N per turn.

7. The sensor according to any of claims 1 to 6, characterized in that the second detection section (25) has at least one transverse dimension (D1) of the fiber smaller than at least one transverse dimension (D2) of the fiber in the first and third sections (23, 27).

8. The sensor according to any of the preceding claims, characterized in that the second fiber section (25) has a fiber diameter (D1) of between 50 and 450 micrometers.

9. The sensor according to any of claims 1 to 6, characterized in that the fiber on the first, second and third sections (23, 25, 27) has a fiber thickness of between 50 and 450 micrometers that is constant over these first, second and third sections (23, 25, 27).

10. The sensor according to any of the preceding claims, characterized in that the first and third fiber sections (23, 27) are inserted in a protective sheath (40), the second section (25) projecting at least partly from one end (43) of the sheath and being intended to come into contact with the medium being examined, whether this be solid and/or liquid and/or gaseous.

11. The sensor according to any of the preceding claims, characterized in that the first and third fiber sections (23, 27) are inserted in an operating channel of a medical diagnosis device (40), the second section (25) projecting at least in part from one end (43) of the channel and being intended to come into contact with a tissue or biological fluid in vivo and/or ex vivo.

12. The sensor according to any of the preceding claims, characterized in that the proportion of X by weight is equal to or higher than 10% and equal to or lower than 70%, whilst the proportion of Y by weight is equal to or higher than 30% and equal to or lower than 90%.

13. A method for fabricating said sensor according to any of the preceding claims, characterized in that
the chalcogenide fiber (2) has a composition of XY type where X is chosen from among Ge, As or Sb or a mixture of two or more than two of these components, and where Y is chosen from among S, Se, Te or a mixture of two or more than two of these components, the chalcogenide fiber (2) having a glass transition temperature T_{g}, the fiber successively comprising over its length between two first and second ends (21, 22) of the fiber (2) the first fiber section (23), an intermediate part (25) which is to form the second fiber section (25) and the third fiber section (27),
a core (50) is heated having a contact zone (53) of transverse dimensions smaller than 4.6 millimeters, to a certain temperature, and the intermediate part (25) of the fiber (2) is applied against said contact zone of the core (50) so that the intermediate part (25) in contact with the zone (53) of the core (50) has a temperature T₂ with:
1.05·T_{g}≦T₂≦1.5·T_{g},
the intermediate part (25) of the fiber (2) is wound around the contact zone (53) of the core (50) at a winding angle of at least 180° so as to form said bent part (28) in said intermediate part (25) of the fiber forming said second section (25) of the fiber with a radius of curvature locally smaller than 2.3 millimeters.

14. The fabrication method according to claim 13, characterized in that the core (50) comprises first and second portions (51, 52) between which there lies said contact zone (53), the core being heated by at least one of the first and second portions (51, 52).

15. The fabrication method according to any of claims 13 and 14, characterized in that the core (50) is cylindrical with any cross-section.

16. The fabrication method according to any of claims 13 to 15, characterized in that the temperature T₂ exceeds the glass transition temperature T_{g} by 10% to 20%.

17. The fabrication method according to any of claims 13 to 16, characterized in that the intermediate part (25) has a fiber diameter (D2) of between 50 and 450 micrometers.

18. The method according to any of claims 13 to 17, characterized in that the bent part (28) has a mechanical compressive strength, in the direction in which it is sought to draw together two separate points belonging to the bent part (28), which is equal to or higher than 1 N.

19. The sensor according to any of claims 13 to 18, characterized in that the bent part (28) comprises a winding comprising at least one turn,
the bent part (28) having a mechanical compressive strength, in the direction in which it is sought to draw together two separate points belonging to the bent part (28), which is equal to or higher than 1 N per turn.
